(19) United States
(12) Patent Application Publication
(10) Pub. No.: US 2016/0101124 A1
(43) Pub. Date: Apr. 14, 2016

(54) NANO-LIPOSOMAL AMINOGLYCOSIDE-THYMOQUINONE FORMULATIONS

(71) Applicants: KING ABDULLAH INTERNATIONAL MEDICAL RESEARCH CENTER, RIYADH (SA); KING SAUD BIN ABDULAZIZ UNIVERSITY FOR HEALTH SCIENCES, RIYADH (SA); NATIONAL GUARD HEALTH AFFAIRS, RIYADH (SA)

(72) Inventors: MAJED ABDULAZIZ HALWANI, RIYADH (SA); HANAN HASSAN BALKHY, RIYADH (SA)

(21) Appl. No.: 14/513,114

(22) Filed: Oct. 13, 2014

(51) Int. Cl. A61K 31/7036; A61K 45/06; A61K 9/127; A61K 31/122

(52) U.S. Cl. CPC A61K 31/7036; A61K 31/122; A61K 45/06; A61K 9/1277; A61K 9/127

(57) ABSTRACT

The nano-liposomal aminoglycoside-thymoquinone formulations are suitable for administration to a mammal and include an aminoglycoside antibiotic (amikacin, gentamicin, or tobramycin) and thymoquinone (TQ) encapsulated within a liposome. The liposome-encapsulated aminoglycoside-thymoquinone (TQ) formulations can be administered to a subject in need thereof. The liposome-encapsulated aminoglycoside-thymoquinone formulations are prepared by a method, which comprises forming a lipid film from a mixture of phospholipids and cholesterol; mixing the lipid film with methanol containing thymoquinone in molar ratio; evaporating off the methanol from the mixture; adding polysaccharides and/or polyethylene glycol (PEG) in PBS (phosphate-buffered saline) buffer in volume ratio to form a liposomal mixture; sonicating the liposomal mixture for at least five minutes; adding an aminoglycoside antibiotic in molar ratio with the thymoquinone; sonicating the mixture for at least five minutes; lyophilizing the liposomal aminoglycoside-thymoquinone formulation; and storing it at −80° C. until use.

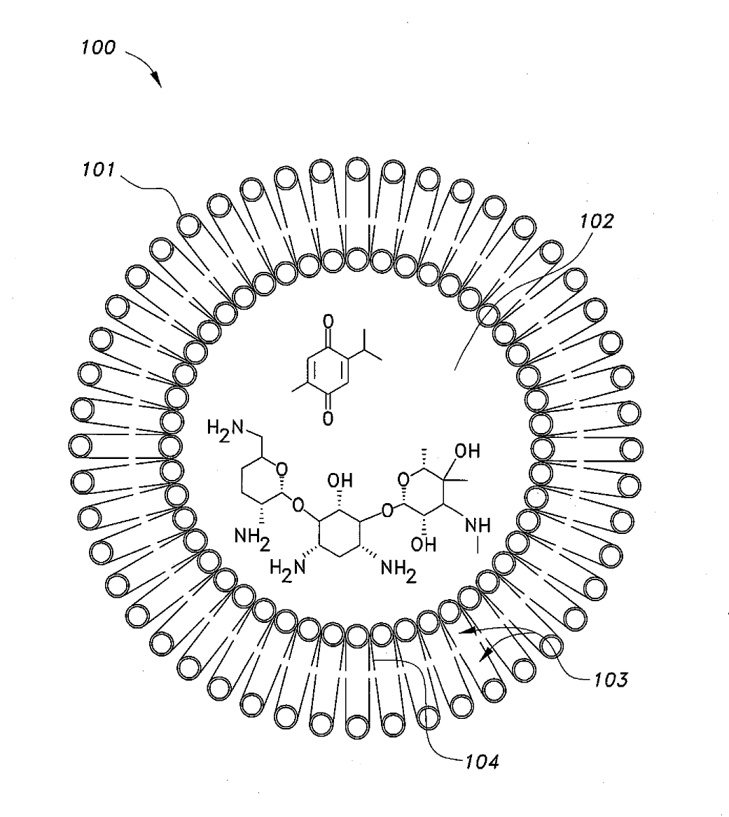

### BACKGROUND OF THE INVENTION

1. Field of the Invention

The present invention relates to antimicrobial compositions, and particularly to a nano-liposomal thymoquinone formulation that provides an aminoglycoside antibiotic (either amikacin, gentamycin (also spelled gentamicin) or tobramycin) and thymoquinone (TQ) encapsulated within the same liposomal vesicle.

2. Description of the Related Art

The aminoglycosides are a family of bactericidal antibiotics that contain amino sugars in glycosidic linkages. Amikacin, gentamicin and tobramycin are all polycationic aminoglycoside antibiotics with a broad spectrum of antibacterial activity. The aminoglycosides are freely soluble in water, and the majority of the drug remains in extracellular locations. They are polycations, and their polarity is primarily responsible for the pharmacokinetic properties shared by the group. The aminoglycosides inhibit protein synthesis in a variety of microorganisms and are useful therapeutically and prophylactically in the treatment of serious, often life-threatening bacterial infections. They are particularly useful in the treatment of infections (e.g., septicemia, peritonitis, pneumonia, urinary tract infections) due to organisms that are resistant to other antibiotics, such as species of Pseudomonas, E. coli, etc.

However, aminoglycosides, like many other antibiotics that are active in vitro, are often inactive against intracellular bacteria in vivo because of its poor penetration into cells. Moreover, the use of aminoglycoside antibiotics is often limited by potentially serious adverse toxicities. Among these are ototoxicity, nephrotoxicity and a potentially fatal neuromuscular paralysis. For example, gentamicin nephrotoxicity causes tubular damage in the kidney upon prolonged gentamicin consumption. Both apoptosis and necrosis cell death phenotypes can be correlated to gentamycin in vivo concentrations. Thus, despite gentamycin's potential bactericidal activity, it is not widely used due to its toxicity. Liposomal encapsulation of aminoglycoside antibiotics has been provided as a means of altering the bio-distribution and efficacy of the drug. However, there is still a long felt need in minimizing the toxicity of these antibiotics, among others. Therefore, it would be desirable to increase aminoglycoside bactericidal activity while lessening its renal toxicity in-vivo.

Thus, nano-liposomal aminoglycoside-thymoquinone formulations solving the aforementioned problems is desired.

### SUMMARY OF THE INVENTION

The nano-liposomal aminoglycoside-thymoquinone formulations are suitable for administration to a mammal and comprise an aminoglycoside antibiotic (either amikacin, gentamycin, or tobramycin) with thymoquinone (TQ) encapsulated within a liposome. The liposome-encapsulated thymoquinone (TQ) formulation can be administered to a subject in need thereof. The liposome-encapsulated aminoglycoside-thymoquinone formulation is prepared by a method, which comprises forming a lipid film from a mixture of phospholipids and cholesterol; mixing the lipid film with methanol containing thymoquinone in molar ratio; evaporating off the methanol from the mixture; adding polysaccharides and/or polyethylene glycol (PEG) in PBS (phosphate-buffered saline) buffer in volume ratio to form a liposomal mixture; sonicating the liposomal mixture for at least five minutes; adding a selected aminoglycoside antibiotic in molar ratio with the thymoquinone; sonicating the mixture for at least five minutes; lyophilizing the liposomal aminoglycoside-thymoquinone formulation; and storing it at −80° C. until use.

These and other features of the present invention will become readily apparent upon further review of the following specification and drawings.

### BRIEF DESCRIPTION OF THE DRAWINGS

The sole drawing FIGURE is a schematic diagram of a liposome encapsulating an aminoglycoside {in this example, gentamicin) and thymoquinone (TQ) within the vesicle.

### DETAILED DESCRIPTION OF THE PREFERRED EMBODIMENTS

The nano-liposomal aminoglycoside-thymoquinone formulations are suitable for administration to a mammal and comprises an aminoglycoside antibiotic (either amikacin, gentamycin, or tobramycin) and thymoquinone (TQ) encapsulated within the same liposome. The liposome-encapsulated aminoglycoside-thymoquinone (TQ) formulation can be administered to a subject in need thereof. The liposome-encapsulated aminoglycoside-thymoquinone formulation is prepared by a method, which comprises forming a lipid film from a mixture of phospholipids and cholesterol; mixing the lipid film with methanol containing thymoquinone in molar ratio; evaporating off the methanol from the mixture; adding polysaccharides and/or polyethylene glycol (PEG) in sucrose in PBS (phosphate-buffered saline) buffer in volume ratio to form a liposomal mixture; sonicating the liposomal mixture for at least five minutes; adding aminoglycoside antibiotic in molar ratio with the thymoquinone; sonicating the mixture for at least five minutes; lyophilizing the liposomal aminoglycoside-thymoquinone formulation; and storing it at −80° C. until use.

As described herein, a “liposome” is a spherical vesicle composed of a unilamellar phase different type phospholipids bilayer. Liposomal vesicles that are able to be assembled inside aquatic milieu exhibit the phenomenon of hydrophilic and hydrophobic forces on phospholipid heads and tails. Hydrophobic tails face each other as shelter from water, whereas the hydrophilic heads face the water, thus forming multi-bilayers that give liposomes a vesicle shape, which can be divided into internal core, niches in-between phospholipids tails, and an external membrane. Therefore, a liposome can entrap various compounds and can be used as a vehicle for the administration of pharmaceutical drugs.

It will be understood that, as used herein, an “aminoglucoside antibiotic” is a molecule that comprises glycidyl residues having NH groups in a side chain. The term, thus, encompasses other molecules than those corresponding to the conventional pharmacological definition of so-called aminoglucoside antibiotics. The antibiotic is preferably an aminoglycoside, such as neomycin, Kanamycin, amikacin, tobramycin, gentamicin, streptomycin, paromomycin, and other members of the aminoglycoside family. Described below is an exemplary method to produce the liposomal gentamicin-thymoquinone (LGTQ) formulations.

### EXAMPLE 1

### The method of Making Liposomal Gentamicin-TQ (LGTQ) Formulations

A dehydration-rehydration technique is used to prepare multi-lamellar nano-vesicle liposomes containing thymoquinone (TQ). Initially, 1,2-Distearoyl-sn-Glycero-3-Phosphocholine (DSPC), 1,2-Dipalmitoyl-sn-Glycero-3-Phosphocholine (DPPC), or 1,2-Dimyristoyl-sn-Glycero-3-Phosphocholine (DMPC) is dissolved individually with cholesterol in chloroform in molar ratio. Then chloroform from the mixture is evaporated off using a rotary evaporator. The resulting lipid film is mixed with methanol containing 2-isopropyl-5-methylbenzo-1,4-quinone, i.e., thymoquinone (TQ) in molar ratio. The rotary evaporator is used again to clear out the methanol from the mixture. Dissolved sucrose in PBS buffer in volume ratio to lipids is added, and the liposomal mixture is sonicated once for 5 minutes, both before and after the addition of gentamicin) or other aminoglycoside antibiotic). The liposomal gentamicin-TQ (LGTQ) formulation with encapsulated drugs is lyophilized and kept at −80° C. until use. In the case of adding amikacin, the formulation is termed Liposomal Amikacin-TQ (LATQ). In the case of adding tobramycin, the formulation is termed Liposomal Tobramycin-TQ (LTTQ).

The antibiotic is preferably an aminoglycoside, such as gentamicin, neomycin, kanamycin, amikacin, tobramycin, sisomicin, netilmicin, streptomycin, paromomycin and other members of the aminoglycoside family. Herein, amikacin, gentamycin and tobramycin are the preferred aminoglycoside antibiotics. Aminoglycoside antibiotics composed of a mixture of related components and fractions that are used to treat many types of bacterial infections, particularly those caused by Gram-negative organisms. Below are the structural formulas of amikacin, gentamicin and tobramycin.

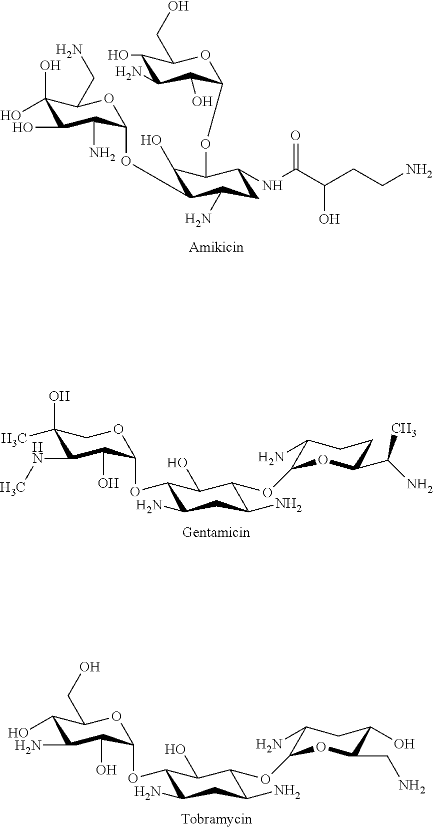

Thymoquinone, 2-isopropyl-5-methylbenzo-1,4-quinone is a phytochemical compound found in the plant Nigella sativa. It has antioxidant effects as well as antibacterial properties. The structure of thymoquinone is shown below.

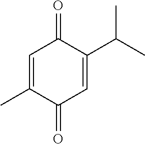

As illustrated in the sole drawing, the liposome formulation 100 comprises a bilayer 103 having a hydrophilic head 101 and a hydrophobic tail 104 pointing away from the hydrophilic core 102. Amikacin, gentamicin, or tobramycin with thymoquinone (TQ) can be encapsulated within the hydrophilic core 102, i.e., gentamycin (or amikacin or tobramycin) and thymoquinone share a common liposome. The liposomal formulation typically comprises a diameter of between 10 nm and 1 micron (1000 nm). The liposome acts as a carrier for the active ingredients, amikacin, gentamicin, or tobramycin antibiotic and thymoquinone (TQ).

The liposomal formulation (LTQ) increases amikacin, gentamicin or tobramycin bioactivity through synergy with the antimicrobial power of TQ and the diffusion properties of the liposomes on bacterial membranes. The liposome vehicle avoids the close contact between amikacin, gentamicin or tobramycin and host kidney cells. Additionally, the TQ is a strong antioxidant that prevents gentamicin renal toxicity, thereby reducing the risk of acute renal failure.

The liposomal/aminoglycoside formulations prepared according to the method described above exhibit encapsulation efficiencies that ranged from ≦5-60% without alteration of in vitro biological activity.

A pharmaceutical composition of a liposome/antibiotic suspension prepared according to the above method can be administered intravenously, locally, topically, etc. in a dose which varies according to the manner of administration, the drug being delivered, and the stage of the infection or other condition being treated.

Depending on the intended mode of administration and the intended use, the compositions may be in the form of a powder, in forms for inhalation, or in liquid or semi-solid dosage forms for oral uptake or as pastes, creams, ointments, and emulsions for external use, and may be in unit-dosage forms of capsules and tablets suitable for oral administration. The LTQ formulations may include a conventional pharmaceutical carrier or excipient and, in addition, may include other medicinal agents, antibiotics, growth factors, etc.

The nano-liposomal gentamycin-thymoquinone formulations may be administered to a warm-blooded animal, such as humans or other mammals already suffering from an infection in an amount sufficient to reduce, terminate, or significantly inhibit the progression of the infection. Amounts adequate to accomplish these effects are defined as a “therapeutically effective doses”. Amounts effective for this use will depend on the severity of the infection and its site, and the general susceptibility of the bacterium to the antibiotic being used, e.g., gentamicin, and the general state of health of the patient being treated.

The amount of drug administered via the liposomal/drug (LTQ) formulations described herein can also be increased above those typically used due to the minimization of toxicity to the patient and the overall increased therapeutic effectiveness of the preparations, as illustrated herein below, as might be necessary in the case of severe, life-threatening infections. Maintenance dosages over a prolonged period of time may be adjusted as necessary. For veterinary uses in animals other than humans, higher levels may also be administered as necessary. Determining actual amounts of the liposomal/drug complexes necessary to treat a particular condition as described above will be through standard empirical methods well known in the art.

The LTQ formulations can be used in combination with other drugs, including other antibiotics, found to improve treatment responses. In this manner, a synergistic effect may be attained that yields a clinical efficacy greater than that realized with any single antibiotic.

Because of the liposomal encapsulation, the gentamicin toxicity is reduced owing to reduced gentamicin close contact with renal epithelial cells. Additionally, the LGTQ formulation will work in parallel to increase gentamicin penetration on bacterial cell membrane owing to the liposomal properties. Importantly, the encapsulation of thymoquinone (TQ) together with gentamicin in one liposomal formulation will work on preventing gentamicin toxicity due to the capability of TQ to reverse gentamicin renal toxicity. Given that TQ is also an antimicrobial agent, the combined gentamicin TQ formulation would display greatly enhanced antibacterial activity. In addition, since gentamicin and thymoquinone are encapsulated in a common liposome, the gentamicin and the thymoquinone may be released simultaneously, which might not be the case if the gentamicin and thymoquinone are encapsulated in different liposomes. Notably, theoretically thymoquinone could have the same effect on amikacin and tobramycin due to their chemical structure similarity with gentamicin.

It is to be understood that the present invention is not limited to the embodiments described above, but encompasses any and all embodiments within the scope of the following claims.

We claim:

1. A composition, comprising an aminoglycoside antibiotic and thymoquinone encapsulated within a common liposome.

2. The composition according to claim 1, wherein said aminoglycoside antibiotic is selected from the group consisting of amikacin, gentamicin, and tobramycin.

3. The composition of claim 1, further comprising an antimicrobial, antiparasitic, antiviral or anticancer agent encapsulated within the liposome with the aminoglycoside antibiotic and the thymoquinone.

4. The composition according to claim 1, wherein said liposome comprises:
at least one phospholipid; and
cholesterol.

5. The composition according to claim 1, said liposome has a diameter between 10 nm and 1 micron.

6. A method of treating a mammal, comprising the step of administering a therapeutically effect amount of a composition including at least one aminoglycoside antibiotic selected from the group consisting of amikacin, gentamicin and tobramycin and thymoquinone encapsulated in a liposomal carrier to the mammal.

7. A process for preparing a liposomal aminoglycoside-thymoquinone formulation suitable for administration to a mammal, comprising the steps of:
forming a lipid film from a mixture of phospholipids and cholesterol;
mixing the lipid film with methanol containing thymoquinone in molar ratio of lipid to thymoquinone;
evaporating the methanol from the mixture;
adding polysaccharides and/or Polyethylene Glycol (PEG) in phosphate buffered saline buffer in volume ratio to form a liposomal mixture;
sonicating the liposomal mixture for at least five minutes;
adding selected aminoglycoside antibiotic in molar ratio with the thymoquinone;
sonicating the mixture for at least five minutes;
lyophilizing the liposomal aminoglycoside thymoquinone formulation; and
storing the formulation at −80° C. until use.

8. The process for preparing a liposomal aminoglycoside-thymoquinone formulation according to claim 7, wherein said aminoglycoside antibiotic is selected from the group consisting of amikacin, gentamicin and tobramycin.
